(19) United States
(12) Patent Application Publication
(10) Pub. No.: US 2019/0021674 A1
(43) Pub. Date: Jan. 24, 2019

(54) Methods for Treating Cholesterol-Related Diseases

(71) Applicant: HDL Therapeutics, Inc., Vero Beach, FL (US)

(72) Inventors: Hollis Bryan Brewer, Jr., Potomac, MD (US); Michael M. Matin, Short Hills, NJ (US)

(21) Appl. No.: 15/876,808

(22) Filed: Jan. 22, 2018

Related U.S. Application Data
(60) Provisional application No. 62/516,100, filed on Jun. 6, 2017.
(60) Provisional application No. 62/465,262, filed on Mar. 1, 2017.
(60) Provisional application No. 62/449,416, filed on Jan. 23, 2017.

(51) Int. Cl. A61B 5/00; A61F 2/82; A61B 5/02; A61M 1/36; A61L 31/16

(52) U.S. Cl. CPC A61B 5/6866; A61F 2/82; G01N 33/4925; A61M 1/3607; A61L 31/16; A61B 5/02007

(57) ABSTRACT

The present specification is directed to systems, apparatus and methods for treating lipid-related diseases including homozygous familial hypercholesterolemia, heterozygous familial hypercholesterolemia, ischemic stroke, coronary artery disease, acute coronary syndrome, peripheral arterial disease, or renal arterial disease and its complications, and for treating the progression of Alzheimer's disease using imaging techniques to assess changes in one or more lipid-containing atheroma areas and volumes after serial infusions of delipidated plasma as compared to a baseline

### CROSS-REFERENCE

The present application relies on U.S. Provisional Patent Application No. 62/449,416, entitled “Method for Treating Familial Hypercholesterolemia” and filed on Jan. 23, 2017, for priority.

The present application also relies on U.S. Provisional Patent Application No. 62/465,262, entitled “Method for Treating Familial Hypercholesterolemia” and filed on Mar. 1, 2017, for priority.

The present application relies on U.S. Provisional Patent Application No. 62/516,100, entitled “Methods for Treating Cholesterol-Related Diseases” and filed on June 6, 2017, for priority.

The above-mentioned applications are all incorporated herein by reference in their entirety.

### FIELD

The present invention generally relates to systems, apparatus and methods for removing lipids from HDL particles while leaving LDL particles substantially intact, via the extracorporeal treatment of blood plasma using either a single solvent or multiple solvents. The method of the present specification provides for successively repeated treatment procedure for selective removal of lipid from HDL to create a modified HDL particle while leaving LDL particles substantially intact, in order to treat chronic cardiovascular diseases and acute renal diseases.

### BACKGROUND

Familial Hypercholesterolemia (FH) is an inherited genetic autosomal dominant disease characterized by markedly elevated low density lipoprotein (LDL), tendon xanthomas, and premature coronary heart disease, caused by mutations of “FH genes,” which include the LDL-receptor (LDLR), apolipoprotein B-100 (APOB) or proprotein convertase subtilisin/kexin type 9 (PCSK9). FH produces a clinically recognizable pattern that consists of severe hypercholesterolemia due to the accumulation of LDL in the plasma, cholesterol deposition in tendons and skin, as well as a high risk of atherosclerosis manifesting almost exclusively as coronary artery disease (CAD). In FH patients, this genetic mutation makes the liver unable to effectively metabolize (or remove) excess plasma LDL, resulting in increased LDL levels.

If an individual has inherited a defective FH gene from one parent, the form of FH is called Heterozygous FH. Heterozygous FH is a common genetic disorder, inherited in an autosomal dominant pattern, occurring in approximately 1:500 people in most countries. If the individual has inherited a defective FH gene from both parents, the form of FH is called Homozygous FH. Homozygous FH is very rare, occurring in about 1 in 160,000 to one million people worldwide, and results in LDL levels >700 mg/dl, 10 fold higher than the ideal 70 mg/dl level desired for patients with CVD. Due to the high LDL levels, patients with Homozygous FH have aggressive atherosclerosis (narrowing and blocking of blood vessels) and early heart attacks. This process starts before birth and progresses rapidly. It can affect the coronary arteries, carotid arteries, aorta, and aortic valve.

Heterozygous FH (HeFH) is normally treated with statins, bile acid sequestrants, or other lipid lowering agents that lower cholesterol levels, and/or by offering genetic counseling. Homozygous FH (HoFH) often does not respond adequately to medical therapy and may require other treatments, including LDL apheresis (removal of LDL in a method similar to dialysis), ileal bypass surgery to dramatically lower their LDL levels, and occasionally liver transplantation. A few medications have recently been approved for use by HoFH subjects. However, these medications lower LDL only, and modestly contribute to slowing, but not stopping, further progression of atherosclerosis. Additionally, these medications are known to have significant side-effects.

Cholesterol is synthesized by the liver or obtained from dietary sources. LDL is responsible for transferring cholesterol from the liver to tissues at different sites in the body. However, if LDL collects on the arterial walls, it undergoes oxidation caused by oxygen free radicals liberated from the body's chemical processes and interacts deleteriously with the blood vessels. The modified LDL causes white blood cells in the immune system to gather at the arterial walls, forming a fatty substance called plaque and injuring cellular layers that line blood vessels. The modified oxidized LDL also reduces the level of nitric oxide, which is responsible for relaxing the blood vessels and thereby allowing the blood to flow freely. As this process continues, the arterial walls slowly constrict, resulting in hardening of the arteries and thereby reducing blood flow. The gradual build-up of plaque can result in blockage of a coronary vessel and ultimately in a heart attack. The plaque build up can also occur in peripheral vessels such as the legs and this condition is known as peripheral arterial disease.

Obstructions can also appear in blood vessels that supply blood to the brain, which can result in ischemic strokes. The underlying condition for this type of obstruction is the development of fatty deposits lining the vessel walls. It is known that at least 2.7% of men and women over the age of 18 in the United States have a history of stroke. Prevalence of stroke is also known to be higher with increasing age. With the increase in the aging population, the prevalence of stroke survivors is projected to increase, especially among elderly women. A considerable portion of all strokes (at least 87%) are ischemic in nature.

Further, it has been shown that hypercholesterolemia and inflammation are two dominant mechanisms implicated in the development of atherosclerosis. There is significant overlap between vascular risk factors for both Alzheimer's disease and atherosclerosis. Inflammation has been implicated in Alzheimer's disease pathogenesis and it is suggested that abnormalities in cholesterol homeostasis may have a role as well. In addition, many of the contributory factors in atherogenesis also contribute to Alzheimer's disease. Specifically, in cell cultures, increased and decreased cholesterol levels promote and inhibit the formation of beta amyloid (Aβ) from Amyloid Precursor Protein (APP), respectively. Thus, the use of treatments with proven effects on the process of atherosclerosis may be one method for treating the progression of the Alzheimer's disease.

Another common cardiovascular disease that occurs due to development of atherosclerosis (hardening and narrowing of the arteries) within the elastic lining inside a coronary artery, is Coronary Artery Disease (CAD), also known as Ischemic Heart Disease (IHD). On the basis of a statistical data collected from 2009 to 2012, an estimated 15.5 million Americans ≥20 years of age have CAD. The total CAD prevalence in the United States is 6.2% of adults ≥20 years of age.

An accurate decrease in blood flow in the coronary arteries may result in part of the heart muscle unable to function properly. This condition is known as Acute Coronary Syndrome (ACS). A conservative estimate for the number of hospital discharges with ACS in 2010 is 625,000.

In contrast to LDL, high plasma HDL levels are desirable because they play a major role in “reverse cholesterol transport”, where the excess cholesterol is transferred from tissue sites to the liver where it is eliminated. Optimal total cholesterol levels are 200 mg/dl or below with a LDL cholesterol level of 160 mg/dl or below and a HDL-cholesterol level of 45 mg/dl for men and 50 mg/dl for women. Lower LDL levels are recommended for individuals with a history of elevated cholesterol, atherosclerosis or coronary artery disease. High levels of LDL increase the lipid content in coronary arteries resulting in formation of lipid filled plaques that are vulnerable to rupture. On the other hand, HDL has been shown to decrease the lipid content in the lipid filled plaques, reducing the probability of rupture. In the last several years, clinical trials of low density lipoprotein (LDL)-lowering drugs have definitively established that reductions in LDL are associated with a 30-45% decrease in clinical cardiovascular disease (CVD) events. CVD events include events occurring in diseases such as HoFH, HeFH, and peripheral arterial disease. Despite lowered LDL, however, many patients continue to have cardiac events. Low levels of HDL are often present in high risk subjects with CVD, and epidemiological studies have identified HDL as an independent risk factor that modulates CVD risk. In addition to epidemiologic studies, other evidence suggests that raising HDL would reduce the risk of CVD. There has been increasing interest in changing plasma HDL levels by dietary, pharmacological or genetic manipulations as a potential strategy for the treatment of CVD including HoFH, HeFH, Ischemic stroke, CAD, ACS, and peripheral arterial disease and for treating the progression of Alzheimer's Disease.

The protein component of LDL, known as apolipoprotein-B (ApoB), and its products, comprise atherogenic elements. Elevated plasma LDL levels and reduced HDL levels are recognized as primary causes of coronary disease. ApoB is in highest concentration in LDL particles and is not present in HDL particles. Apolipoprotein A-I (ApoA-I) and apolipoprotein A-II (ApoA-II) are found in HDL. Other apolipoproteins, such as ApoC and its subtypes (C-I, C-II and C-III), ApoD, and ApoE are also found in HDL. ApoC and ApoE are also observed in LDL particles.

Numerous major classes of HDL particles including HDL2b, HDL2a, HDL3a, HDL3b and HDL3 have been reported. Various forms of HDL particles have been described on the basis of electrophoretic mobility on agarose as two major populations, a major fraction with α-HDL mobility and a minor fraction with migration similar to VLDL. This latter fraction has been called pre-β HDL and these particles are the most efficient HDL particle subclass for inducing cellular cholesterol efflux.

The HDL lipoprotein particles are comprised of ApoA-I, phospholipids and cholesterol. The pre-β HDL particles are considered to be the first acceptors of cellular free cholesterol and are essential in eventually transferring free and esterified cholesterol to α-HDL. Pre-β HDL particles may transfer cholesterol to α-HDL or be converted to α-HDL. The alpha HDL transfers cholesterol to the liver, where excess cholesterol can be removed from the body.

HDL levels are inversely correlated with atherosclerosis and coronary artery disease. Once cholesterol-carrying α-HDL reaches the liver, the α-HDL particles divest of the cholesterol and transfer the free cholesterol to the liver. The α-HDL particles (divested of cholesterol) are subsequently converted to pre-β HDL particles and exit the liver, which then serve to pick up additional cholesterol within the body and are converted back to α-HDL, thus repeating the cycle. Accordingly, what is needed is a method to decrease or remove cholesterol from these various HDL particles, especially the α-HDL particles, so that they are available to remove additional cholesterol from cells.

Renal arterial stenosis refers to a blockage in an artery that supplies blood to the kidney and is characterizes in two forms: a) smooth muscle plaque or b) cholesterol filled plaque. This condition, generally known as renal arterial stenosis, decreases blood flow to the kidney and can result in high blood pressure. Plaque in the renal arteries may be discovered during a CT angiogram. In some cases, renal arterial stenosis is discovered while performing a CT angiogram for an aortic aneurysm. Conventionally, blood pressure increases gradually with age. However, a sudden onset of hypertension is also likely to be associated with renal obstruction or renal arterial stenosis. A decrease in flow of blood to the kidney causes vasoconstriction or high blood pressure, as the kidney starts producing an excess of cytokines.

In addition, a “cholesterol embolism”, can occur when the cholesterol in the artery is released, usually from an atherosclerotic plaque, and travels as an embolus in the bloodstream causing an obstruction (as an embolism) in blood vessels that are positioned further away. Once in circulation, the cholesterol particles get stuck in tiny blood vessels, or arterioles. They can reduce blood flow to tissues and cause inflammation and tissue damage that can harm the kidneys. A cholesterol embolism may result in renal failure, and is a disease state referred to as Atheroembolic Renal Disease (AERD). AERD is one of the manifestations of diseases that may occur due to a cholesterol-filled plaque. In a patient with AERD, the plaque may rupture in the artery and release the cholesterol and other “junk” within the plaque into the vessel. The released cholesterol and junk may travel down the artery and may block the artery and injure a part of the kidney and its tissues, thereby resulting in AERD. Atherosclerosis of the aorta is the most common cause of AERD.

Currently, treatment of renal arterial stenosis, its manifestations such as AERD, and other cardiovascular diseases involves putting a stent in an artery to open the vessel. This technique often normalizes the blood pressure. However, installing a stent is likely to only treat the symptoms, such as high blood pressure. There are also instances when the blood pressure is normal, but AERD is present in a patient. There is thus a need to address the underlying cause of the disease, and treat renal arterial stenosis either in combination with or independently of high blood pressure symptoms.

Hyperlipidemia (or abnormally high concentration of lipids in the blood) may be treated by changing a patient's diet. However, diet as a primary mode of therapy requires a major effort on the part of patients, physicians, nutritionists, dietitians, and other health care professionals and thus undesirably taxes the resources of health professionals. Another negative aspect of this therapy is that its success does not rest exclusively on diet. Rather, success of dietary therapy depends upon a combination of social, psychological, economic, and behavioral factors. Thus, therapy based only on correcting flaws within a patient's diet, is not always successful.

In instances when dietary modification has been unsuccessful, drug therapy has been used as adjunctive therapy. Such therapy has included use of commercially available hypolipidemic drugs administered alone or in combination with other therapies as a supplement to dietary control. These drugs, called statins, include lovastatin, pravastatin, simvastatin, fluvastatin, atorvastatin, and cerivastatin. Statins are particularly effective for lowering LDL levels and are also effective in the reduction of triglycerides, apparently in direct proportion to their LDL-lowering effects. Statins raise HDL levels, but to a lesser extent than other anti-cholesterol drugs. Statins also increase nitric oxide, which, as described above, is reduced in the presence of oxidized LDL.

Bile acid resins, another drug therapy, work by binding with bile acid, a substance made by the liver using cholesterol as one of the primary manufacturing components. Because the drugs bind with bile acids in the digestive tract, they are then excreted with the feces rather than being absorbed into the body. The liver, as a result, must take more cholesterol from the circulation to continue constructing bile acids, resulting in an overall decrease in LDL levels.

Nicotinic acid, or niacin, also known as vitamin B3, is effective in reducing triglyceride levels and raising HDL levels higher than any other anti-cholesterol drug. Nicotinic acid also lowers LDL-cholesterol.

Fibric acid derivatives, or fibrates, are used to lower triglyceride levels and increase HDL when other drugs ordinarily used for these purposes, such as niacin, are not effective.

Probucol lowers LDL-cholesterol levels, however, it also lowers HDL levels. It is generally used for certain genetic disorders that cause high cholesterol levels, or in cases where other cholesterol-lowering drugs are ineffective or cannot be used.

PCSK9s lower LDL-cholesterol levels via increasing the cellular level of LDL receptors that reside in the liver.

Hypolipidemic drugs have had varying degrees of success in reducing blood lipid; however, none of the hypolipidemic drugs successfully treats all types of hyperlipidemia. While some hypolipidemic drugs have been fairly successful, the medical community has found little conclusive evidence that hypolipidemic drugs cause regression of atherosclerosis. In addition, all hypolipidemic drugs have undesirable side effects. As a result of the lack of success of dietary control, drug therapy and other therapies, atherosclerosis remains a major cause of death in many parts of the world.

New therapies have been used to reduce the amount of lipid in patients for whom drug and diet therapies were not sufficiently effective. For example, extracorporeal procedures like plasmapheresis and LDL-apheresis have been employed and are shown to be effective in lowering LDL.

Plasmapheresis therapy or plasma exchange therapy, involves replacing a patient's plasma with donor plasma or more usually a plasma protein fraction. Plasmapheresis is a process whereby the blood plasma is removed from blood cells by a cell separator. The separator works either by spinning the blood at high speed to separate the cells from the fluid or by passing the blood through a membrane with pores so small that only the fluid component of the blood can pass through. The cells are returned to the person undergoing treatment, while the plasma is discarded and replaced with other fluids.

This treatment has resulted in complications due to the introduction of foreign proteins and transmission of infectious diseases. Further, plasmapheresis has the disadvantage of non- selective removal of all serum lipoproteins, such as VLDL, LDL, and HDL. Moreover, plasmapheresis can result in several side effects including allergic reactions in the form of fever, chills, and rash and possibly even anaphylaxis.

As described above, it is not desirable to remove HDL, which is secreted from both the liver and the intestine as nascent, disk-shaped particles that contain cholesterol and phospholipids. HDL is believed to play a role in reverse cholesterol transport, which is the process by which excess cholesterol is removed from tissues and transported to the liver for reuse or disposal in the bile.

In contrast to plasmapheresis, the LDL-apheresis procedure selectively removes ApoB containing cholesterol, such as LDL, while retaining HDL.

Several methods for LDL-apheresis have been developed. These techniques include absorption of LDL in heparin-agarose beads, the use of immobilized LDL-antibodies, cascade filtration absorption to immobilize dextran sulfate, and LDL precipitation at low pH in the presence of heparin. Each method described above is effective in removing LDL. This treatment process has disadvantages, however, including the failure to positively affect HDL or to cause a metabolic shift that can enhance atherosclerosis and other cardiovascular diseases. LDL apheresis, as its name suggests, merely treats LDL in patients with severe hyperlipidemia.

Yet another method of achieving a reduction in plasma cholesterol in homozygous familial hypercholesterolemia, heterozygous familial hypercholesterolemia and patients with acquired hyperlipidemia is an extracorporeal lipid elimination process, referred to as cholesterol apheresis. In cholesterol apheresis, blood is withdrawn from a patient, the plasma is separated from the blood, and the plasma is mixed with a solvent mixture. The solvent mixture extracts lipids from the plasma. Thereafter, the delipidated plasma is recombined with the patient's blood cells and returned to the patient. Using this procedure, however, results in a modification of the LDL particles, such that the modified LDL particles could result in increased intensity of the heart disease. At the same time, this process also resulted in further delipidation of the HDL particles.

Conventional extracorporeal delipidation processes, however, are directed toward the concurrent delipidation of LDL and HDL. This process can have a number of disadvantages, mainly in that delipidated LDL tends to aggregate and subsequently cause an increase in heart disease conditions, rather than decrease. In addition, extracorporeal systems are designed to subject body fluid volumes to substantial processing, possibly through multiple stage solvent exposure and extraction steps.

Vigorous multi-stage solvent exposure and extraction can have several drawbacks. It may be difficult to remove a sufficient amount of solvents from the delipidated plasma in order for the delipidated plasma to be safely returned to a patient.

Hence, existing apheresis and extracorporeal systems for treatment of plasma constituents suffer from a number of disadvantages that limit their ability to be used in clinical applications. A need exists for improved systems, apparatuses and methods capable of removing lipids from blood components in order to provide treatments and preventative measures for chronic cardiovascular diseases. Methods have also been provided to selectively remove lipid from HDL particles and thereby create modified HDL particles with increased capacity to accept cholesterol.

While the methods to selectively delipidate HDL particles overcomes several of the limitations stated above, what is also needed is a method to selectively remove lipid from HDL particles and thereby create modified HDL particles with increased capacity to accept cholesterol, without substantially affecting LDL particles, in chronic diseases. What is also needed is a method to successively monitor effectiveness of the modified HDL particles in accepting cholesterol in order to monitor the progress of a treatment using imaging techniques such as CT Angiography.

### SUMMARY

The following embodiments and aspects thereof are described and illustrated in conjunction with systems, tools and methods, which are meant to be exemplary and illustrative, not limiting in scope.

The present specification discloses a method for treatment of cardiovascular disease in a patient, comprising: monitoring changes in one or more blood vessels in the patient; based on said monitoring, determining if lipid-containing degenerative material is present in said one or more blood vessels; monitoring a degree of blood oxygen delivery; based on said determination of lipid-containing degenerative material and degree of blood oxygen delivery, determining a treatment protocol for said cardiovascular disease, wherein the treatment protocol comprises at least one of a placement of a stent in the patient, an administration to the patient of a composition derived from mixing a blood fraction of the patient with a lipid removing agent, or a placement of a stent in the patient together with an administration to the patient of a composition derived from mixing a blood fraction of the patient with a lipid removing agent.

Optionally, the composition is derived by obtaining the blood fraction from the patient; mixing said blood fraction with the lipid removing agent to yield modified high-density lipoproteins; separating said modified high-density lipoproteins; and delivering said modified high-density lipoproteins to said patient.

Optionally, the method comprises: connecting the patient to a device for withdrawing blood; withdrawing blood from the patient; and separating blood cells from the blood to yield the blood fraction containing high density lipoproteins and low density lipoproteins.

Optionally, the modified high density lipoproteins have an increased concentration of pre-beta high density lipoprotein relative to high density lipoproteins from the blood fraction prior to mixing.

Optionally, the degree of blood oxygen delivery is monitored by measuring the patient's fractional flow reserve. The treatment protocol may be determined to be the placement of the stent in the patient if the patient's fractional flow reserve is within a first range of values. The first range of values may be 1% to 79%. The treatment protocol may be determined to be the administration of the composition derived from mixing the blood fraction of the patient with the lipid removing agent if the patient's fractional flow reserve is within a second range of values and if the lipid-containing degenerative material occupies a cross sectional area of the one or more blood vessels that is within a third range of values. The second range of values may be 80%-100% and the third range of values may be 20% to 70%.

Optionally, the cardiovascular disease is at least one of homozygous familial hypercholesterolemia, heterozygous familial hypercholesterolemia, ischemic stroke, coronary artery disease, acute coronary syndrome, or peripheral arterial disease.

The present specification also discloses a method for treatment of a lipid-related disease in a patient, comprising: administering a diagnostic procedure to the patient configured to monitor one or more blood vessels; determining a presence of lipid-containing degenerative material in the one or more blood vessels; identifying an extent of the presence of lipid-containing degenerative material and comparing said extent to a predetermined lipid-containing degenerative material range of values; identifying a level of fractional flow reserve (FFR) and comparing said level to a predetermined threshold FFR range of values; proceeding with a first treatment protocol if said extent of the presence of lipid-containing degenerative material is within a first range and said FFR level is within a second range; proceeding with a second treatment protocol if said FFR level is within a third range that is less than the FFR level within a second range; proceeding with a third treatment protocol if said extent of the presence of lipid-containing degenerative material is within a fourth range that is less than the first range and if said FFR level is within the first range; and proceeding with a fourth treatment protocol if said extent of the presence of lipid-containing degenerative material is within a fifth range that is greater than the first range and if said FFR level is within the first range, wherein each of the first treatment protocol, second treatment protocol, third treatment protocol, and fourth treatment protocol are different.

The extent of the presence of lipid-containing degenerative material may be within 20% to 70% in a first range. The FFR level may be within 80% to 100% in a second range or within 1% to 79% in a second range. The extent of the presence of lipid-containing degenerative material may be within 1% to 19% in a fourth range. The extent of the presence of lipid-containing degenerative material may be within 71% to 100% in a fifth range.

Optionally, the first treatment protocol is an administration to the patient a composition derived from mixing a blood fraction of the patient with a lipid removing agent without a placement of a stent in the patient.

Optionally, the second treatment protocol is a placement of a stent in the patient without an administration to the patient of a composition derived from mixing a blood fraction of the patient with a lipid removing agent.

Optionally, the fourth treatment protocol is selected from either the first treatment protocol or the third protocol, wherein the third protocol is no treatment.

Optionally, the composition is derived by: obtaining the blood fraction from the patient; mixing said blood fraction with the lipid removing agent to yield modified high-density lipoproteins; separating said modified high-density lipoproteins; and delivering said modified high-density lipoproteins to said patient.

Optionally, the lipid-related disease is at least one of homozygous familial hypercholesterolemia, heterozygous familial hypercholesterolemia, ischemic stroke, coronary artery disease, acute coronary syndrome, renal arterial stenosis, peripheral arterial disease, or atheroembolic renal disease.

The present specification also discloses a method for treatment of cardiovascular disease in a patient, comprising: periodically monitoring changes in one or more lipid-containing atheroma areas and volumes in the patient; treating cardiovascular diseases based on the monitoring of the one or more lipid-containing atheroma areas and volumes, the treating comprising: obtaining a blood fraction containing high density lipoprotein and low density lipoprotein from the patient; mixing the blood fraction with a lipid removing agent which removes lipids associated with the high density lipoprotein without substantially modifying the low density lipoprotein to yield a mixture of lipid, the lipid removing agent, modified high density lipoprotein, and the low density lipoprotein; separating the modified high density lipoprotein and the low density lipoprotein from the lipid and the lipid removing agent; and delivering the modified high density lipoprotein and the low density lipoprotein to the patient.

Optionally, the method for treatment of cardiovascular diseases includes method for treatment of at least one of Homozygous Familial Hypercholesterolemia, Heterozygous Familial Hypercholesterolemia, Ischemic stroke, Coronary Artery Disease, Acute Coronary Syndrome, and peripheral arterial disease.

Optionally, the treating cardiovascular diseases based on the monitoring of the one or more atheroma areas and volumes comprises treating if the monitoring determines accumulated lipid-containing degenerative material to be above a predetermined threshold.

Optionally, the treating cardiovascular diseases based on the monitoring of the one or more atheroma areas and volumes comprises treating if the monitoring determines accumulated lipid containing degenerative material to be in the range of 20% to 70%.

Optionally, the periodically monitoring changes comprises monitoring changes within a period of three to six months.

Optionally, the mixing the blood fraction with a lipid removing agent yields modified high density lipoprotein that has an increased concentration of pre-beta high density lipoprotein relative to total protein.

Optionally, the treating cardiovascular diseases further comprises: connecting the patient to a device for withdrawing blood; withdrawing blood containing blood cells from the patient; and separating the blood cells from the blood to yield the blood fraction containing high density lipoprotein and low density lipoprotein.

The present specification also discloses a method for treatment of cardiovascular disease in a patient, comprising: administering a diagnostic procedure to a said patient configured to monitor one or more atheroma; determining the presence of degenerative material; identifying an extent of presence of degenerative material and comparing said extent to a predetermined degenerative material threshold value; identifying a level of Fractional Flow Reserve (FFR) and comparing said level to a predetermined threshold FFR value; proceeding with a delipidation process and implanting a stent in a coronary artery of said patient if said extent of presence of degenerative material is above said predetermined degenerative material threshold value and said FFR level is above said predetermined threshold FFR value; proceeding with a delipidation process if said extent of presence of degenerative material is above said predetermined degenerative material threshold value and said FFR level is below said predetermined threshold FFR value; proceeding with implanting a stent in a coronary artery of said patient if said extent of presence of degenerative material is below said predetermined degenerative material threshold value and said FFR level is above said predetermined threshold FFR value; and providing no treatment if said extent of presence of degenerative material is below said predetermined degenerative material threshold value and said FFR level is below said predetermined threshold FFR value.

Optionally, said predetermined threshold FFR value is equal to 80%.

Optionally, said predetermined threshold degenerative material value is equal to 20%.

Optionally, said delipidation process comprises the steps of: obtaining a blood fraction;

mixing said blood fraction with a lipid removing agent to yield modified high-density lipoproteins (HDL); separating said modified HDL; and delivering said modified HDL to said patient.

The present specification also discloses a method for treatment of Renal Arterial Stenosis (RAS) in a patient, comprising: periodically monitoring changes in one or more lipid-containing atheroma areas and volumes in the patient; treating RAS based on the monitoring of the one or more lipid-containing atheroma areas and volumes, the treating comprising: obtaining a blood fraction containing high density lipoprotein and low density lipoprotein from the patient; mixing the blood fraction with a lipid removing agent which removes lipids associated with the high density lipoprotein without substantially modifying the low density lipoprotein to yield a mixture of lipid, the lipid removing agent, modified high density lipoprotein, and the low density lipoprotein; separating the modified high density lipoprotein and the low density lipoprotein from the lipid and the lipid removing agent; and delivering the modified high density lipoprotein and the low density lipoprotein to the patient.

Optionally, the treating RAS based on the monitoring of the one or more atheroma areas and volumes comprises treating if the monitoring determines accumulated lipid-containing degenerative material to be above a predetermined threshold.

Optionally, the treating RAS based on the monitoring of the one or more atheroma areas and volumes comprises treating if the monitoring determines accumulated lipid containing degenerative material to be in the range of 20% to 70%.

Optionally, the periodically monitoring changes comprises monitoring changes within a period of three to six months.

Optionally, the mixing the blood fraction with a lipid removing agent yields modified high density lipoprotein that has an increased concentration of pre-beta high density lipoprotein relative to total protein.

Optionally, the treating RAS further comprises: connecting the patient to a device for withdrawing blood; withdrawing blood containing blood cells from the patient; and separating the blood cells from the blood to yield the blood fraction containing high density lipoprotein and low density lipoprotein.

The aforementioned and other embodiments of the present specification shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1A is a flow chart delineating the steps of treating cardiovascular diseases using the treatment systems and methods in accordance with embodiments of the present specification;

FIG. 1B is another flow chart delineating the steps of treating cholesterol-related diseases, such as Atheroembolic Renal Disease (AERD), using the treatment systems and methods in accordance with embodiments of the present specification;

FIG. 1C is a table illustrating the types of treatments that may be provided for different compositions of degenerative material determined from an analysis, in accordance with some embodiments of the present specification;

FIG. 2 is a schematic representation of a plurality of components used in accordance with some embodiments of the present specification to achieve the processes disclosed herein; and,

FIG. 3 is a pictorial illustration of an exemplary embodiment of a configuration of a plurality of components used in accordance with some embodiments of the present specification to achieve the processes disclosed herein.

### DETAILED DESCRIPTION

The present specification relates to methods and systems for treating cholesterol-related diseases. Embodiments of the present specification monitor changes in one or more atheroma areas and volumes in a patient, regularly over a period of time. Atheroma areas and volumes are monitored using known imaging techniques, for lipid-containing degenerative material in stenosis.

In accordance with embodiments of the present specification, based on the results of the monitoring, treatment is provided if accumulated lipid-containing degenerative material is identified to be present and above a threshold value. The treatment is repeated each time the atheroma areas and volumes are monitored, at pre-defined time intervals, and accumulated lipid-containing degenerative material is identified to be present and above the threshold.

Embodiments of the present specification treat the condition through systems, apparatuses and methods useful for removing lipid from α-High Density Lipoprotein (α-HDL) particles derived primarily from plasma of the patient thereby creating modified HDL particles with reduced lipid content, particularly reduced cholesterol content. Embodiments of the present specification create these modified HDL particles with reduced lipid content without substantially modifying LDL particles. Embodiments of the present specification modify original α-HDL particles to yield modified HDL particles that have an increased concentration of pre-β HDL relative to the original HDL.

Further, the newly formed derivatives of HDL particles (modified HDL) are administered to the patient to enhance cellular cholesterol efflux and treat cardiovascular diseases and/or other lipid-associated diseases, including Atheroembolic Renal Disease (AERD). The regular periodic monitoring and treatment process renders the methods and systems of the present specification more effective in treating cardiovascular diseases including Homozygous Familial Hypercholesterolemia (HoFH), Heterozygous Familial Hypercholesterolemia (HeFH), Ischemic stroke, Coronary Artery Disease (CAD), Acute Coronary Syndrome (ACS), peripheral arterial disease (PAD), Renal Arterial Stenosis (RAS), and for treating the progression of Alzheimer's Disease.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

It should be noted herein that any feature or component described in association with a specific embodiment may be used and implemented with any other embodiment unless clearly indicated otherwise.

The term “fluid” may be defined as fluids from animals or humans that contain lipids or lipid containing particles, fluids from culturing tissues and cells that contain lipids and fluids mixed with lipid-containing cells. For purposes of this invention, decreasing the amount of lipids in fluids includes decreasing lipids in plasma and particles contained in plasma, including but not limited to HDL particles. Fluids include, but are not limited to: biological fluids; such as blood, plasma, serum, lymphatic fluid, cerebrospinal fluid, peritoneal fluid, pleural fluid, pericardial fluid, various fluids of the reproductive system including, but not limited to, semen, ejaculatory fluids, follicular fluid and amniotic fluid; cell culture reagents such as normal sera, fetal calf serum or serum derived from any animal or human; and immunological reagents, such as various preparations of antibodies and cytokines from culturing tissues and cells, fluids mixed with lipid-containing cells, and fluids containing lipid-containing organisms, such as a saline solution containing lipid-containing organisms. A preferred fluid treated with the methods of the present invention is plasma.

The term “lipid” may be defined as any one or more of a group of fats or fat-like substances occurring in humans or animals. The fats or fat-like substances are characterized by their insolubility in water and solubility in organic solvents. The term “lipid” is known to those of ordinary skill in the art and includes, but is not limited to, complex lipid, simple lipid, triglycerides, fatty acids, glycerophospholipids (phospholipids), true fats such as esters of fatty acids, glycerol, cerebrosides, waxes, and sterols such as cholesterol and ergosterol.

The term “extraction solvent” may be defined as one or more solvents used for extracting lipids from a fluid or from particles within the fluid. This solvent enters the fluid and remains in the fluid until removed by other subsystems. Suitable extraction solvents include solvents that extract or dissolve lipid, including but not limited to phenols, hydrocarbons, amines, ethers, esters, alcohols, halohydrocarbons, halocarbons, and combinations thereof. Examples of suitable extraction solvents are ethers, esters, alcohols, halohydrocarbons, or halocarbons which include, but are not limited to di-isopropyl ether (DIPE), which is also referred to as isopropyl ether, diethyl ether (DEE), which is also referred to as ethyl ether, lower order alcohols such as butanol, especially n-butanol, ethyl acetate, dichloromethane, chloroform, isoflurane, sevoflurane (1,1, 1,3, 3,3-hexafluoro-2-(fluoromethoxy) propane-d3), perfluorocyclohexanes, trifluoroethane, cyclofluorohexanol, and combinations thereof.

The term “patient” refers to animals and humans, which may be either a fluid source to be treated with the methods of the present invention or a recipient of derivatives of HDL particles and or plasma with reduced lipid content.

The term “HDL particles” encompasses several types of particles defined based on a variety of methods such as those that measure charge, density, size and immuno-affinity, including but not limited to electrophoretic mobility, ultracentrifugation, immunoreactivity and other methods known to one of ordinary skill in the art. Such HDL particles include but are not limited to the following: α-HDL, pre-β HDL (including pre-β1 HDL, pre-β2 HDL and pre-β3HDL), HDL2 (including HDL2a and HDL2b), HDL3, VHDL, LpA-I, LpA-II, LpA-I/LpA-II (for a review see Barrans et al., Biochemica Biophysica Acta 1300; 73-85,1996). Accordingly, practice of the methods of the present invention creates modified HDL particles. These modified derivatives of HDL particles may be modified in numerous ways including but not limited to changes in one or more of the following metabolic and/or physico-chemical properties (for a review see Barrans et al., Biochemica Biophysica Acta 1300; 73-85,1996); molecular mass (kDa); charge; diameter; shape; density; hydration density; flotation characteristics; content of cholesterol; content of free cholesterol; content of esterified cholesterol; molar ratio of free cholesterol to phospholipids; immuno-affinity; content, activity or helicity of one or more of the following enzymes or proteins: ApoA-I, ApoA-II, ApoD, ApoE, ApoJ, ApoA-IV, cholesterol ester transfer protein (CETP), lecithin; cholesterol acyltransferase (LCAT); capacity and/or rate for cholesterol binding, capacity and/or rate for cholesterol transport.

The term “fractional flow reserve” or “FFR” is used to refer to a measurement of pressure differences across a coronary artery stenosis (a narrowing, usually due to atherosclerosis) to determine the likelihood that the stenosis impedes oxygen delivery to the heart muscle. Fractional flow reserve is defined as the pressure after (distal to) a stenosis relative to the pressure before the stenosis and is presented as an absolute number. An FFR value of 0.70 means that a given stenosis causes a 30% drop in blood pressure. Thus, FFR is used to express the maximal flow down a vessel in the presence of stenosis compared to the maximal flow in the hypothetical absence of stenosis. A decrease in blood flow, which is measured in terms of blood pressure using FFR, results in a decrease in oxygen delivery via blood (blood oxygen delivery).

The term “blockage due to lipid content” is measured in a percentage and is used to refer to the extent of physical blockage in an artery.

### Cardiovascular Diseases

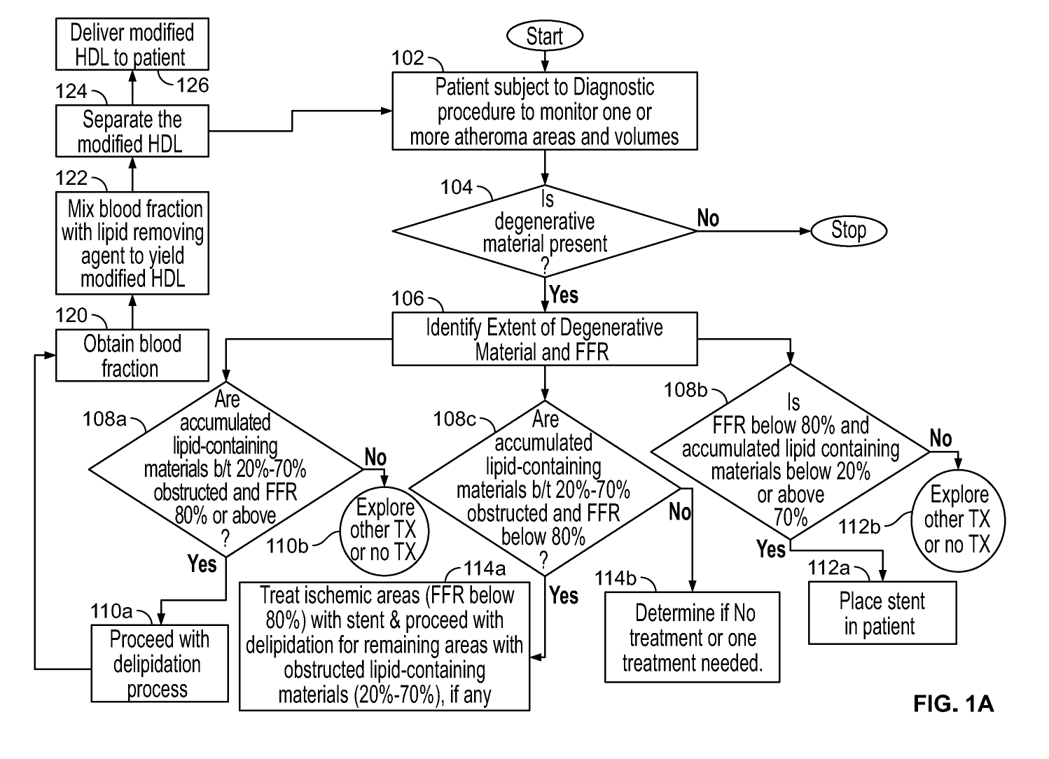

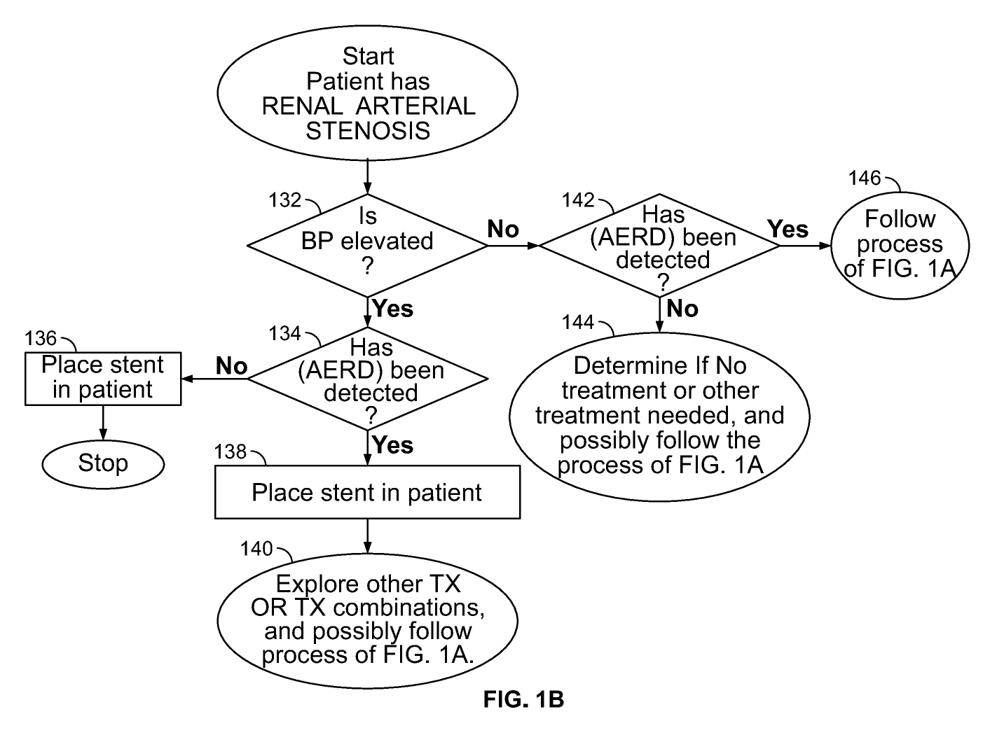

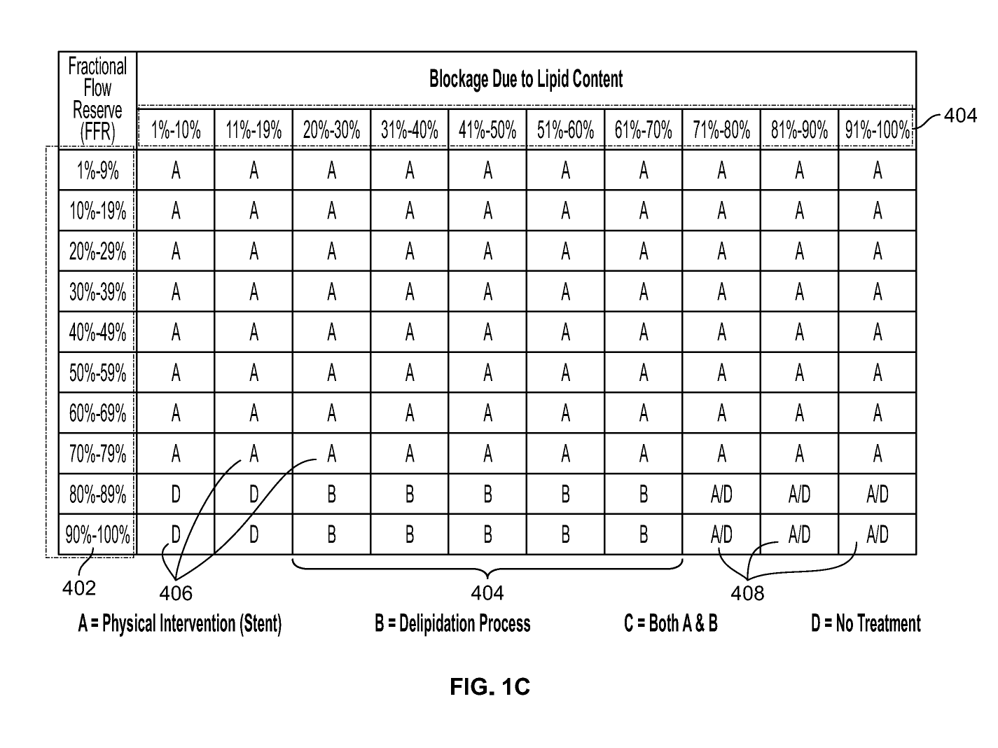

FIG. 1A is a flow chart illustrating an exemplary process of treating cardiovascular diseases, such as, but not limited to HoFH, HeFH, Ischemic stroke, CAD, ACS, peripheral arterial disease (PAD) and for treating the progression of Alzheimer's Disease in accordance with some embodiments of the present specification. At step 102, a subject or a patient who is diagnosed with a cardiovascular disease is monitored for one or more atheroma areas and/or volumes via a diagnostic procedure. In an embodiment, advanced medical imaging techniques, such as, but not limited to Computer Tomography (CT) angiogram and/or Intravascular Ultrasound (IVUS), may be used to detect areas within the inner layer of artery walls where lipid-containing degenerative material may have accumulated. Accumulated degenerative material may include fatty deposits which may include mostly macrophage cells, or debris, containing lipids, calcium and a variable amount of fibrous connective tissue. Analysis from the imaging techniques may also be used to identify and therefore monitor volumes of lipid-containing degenerative material accumulated within the inner layer of artery walls. Lipid-containing degenerative material and non-lipid-containing degenerative material may swell in the artery wall, thereby intruding into the channel of the artery and narrowing it, resulting in restriction of blood flow.

Based on analysis from the diagnostic technique, in step 104, the presence and type of degenerative material is confirmed. In addition, the extent or percentage blockage caused by degenerative material (lipid-containing or non-lipid-containing) is determined by a physician using diagnostic imaging techniques. If no degenerative material is detected at step 104, or if the level of degenerative material falls outside a pre-determined range of values, the process is stopped. In an embodiment, the physician identifies one or more arteries with stenosis that have a blockage of 20%-70% due to accumulated lipids, in order to implement treatment methods in accordance with the present specification. In step 106, a Fractional Flow Reserve (FFR) measurement is used to determine the extent of oxygen delivery in the presence of stenosis. In an embodiment, FFR is used to measure pressure differences across a coronary artery stenosis to determine the likelihood that the stenosis impedes blood oxygen delivery to the heart muscle (ischemia).

Different types of treatments may be provided depending on the diagnostic results and threshold values. At this stage, the physician may determine that either the treatment in accordance with embodiments of the present specification is not required as the disease has subsided, is not present, is not sufficient, or has been treated, or an alternative form of treatment (such as a physical stent) is required.

FIG. 1C is a table illustrating the types of treatments that may be provided for different compositions of degenerative material determined from the diagnosis for cardiovascular diseases, as described in the flow chart of FIG. 1A, in accordance with some embodiments of the present specification. The table compares different types of treatments that may be administered for combinations of various ranges of a Fractional Flow Reserve (FFR) 402, which is indicative of a rate of flow of blood after a blockage (which, in turn, is indicative of blood oxygen delivery), provided in terms of percentage (or fraction) of Fractional Flow Reserve, and various ranges of physical blockage due to lipid content 404, provided in terms of percentage of blockage due to lipid content. Referring to the table, each cell, such as cells 406, corresponds to a combination of a range 402 (indicative of FFR) and a range 404 (indicative of the percentage or extent of blockage due to lipid content), which further indicates at least one method of treatment that may be suitable for that combination.

In embodiments, the different types of treatments are coded as A, B, C, and D. Treatment type ‘A’ corresponds to an invasive treatment process where a stent is embedded through physical intervention. Treatment type ‘B’ corresponds to implementing the treatment methods of selectively modifying HDL particles, in accordance with the embodiments of the present specification. In an embodiment, it is preferable to selectively modify HDL particles (and perform the HDL infusions) where the Fractional Flow Reserve (FFR) ranges from 80-100% and the accumulated lipid obstruction ranges from 20-70%, as noted by sections 404. It should be noted herein that in embodiments, a FFR measurement of 1-79% represents an ischemic condition, wherein a FFR measurement of 80-100% represents a non-ischemic condition. In most cases, treatment types ‘A’ and/or ‘B’ may be able to address the condition. Treatment type ‘D’ corresponds to cases where neither of the stated treatment types (A and/or B) is required. In some cells, such as cells 408, two treatment options may be indicated and the physician would decide upon the appropriate course of treatment.

Treatment type ‘C’ corresponds to cases where a combination of both a stent as well as selective modification of HDL particles is administered (as described in greater detail below with respect to 114a in FIG. 1A). Atherosclerosis is a systemic disease and patients may have multiple lesions throughout their vasculature. Therefore, it should be noted herein that the treatment methods of the present specification are not implemented based on an overall patient health-based treatment strategy, but rather a “lesion/plaque/area/region”-based treatment strategy. Thus, in a few cases, a physician may decide to combine the treatments and administer treatment type ‘C’. If, in a particular patient, one or more areas or lesions have a FFR of 79% or less (ranging from 1% to 79%), then those areas would have a stent implanted. If the same patient presents with additional, remaining lesions that exhibit lipid-based blockage in the range of 20-70% and also an FFR of 80-100%, then the patient would undergo a subsequent delipidation. Therefore, both interventions may be used for patients having multiple lesions with different levels of disease at each lesion.

Referring again to FIG. 1A, at step 108a, a physician determines whether the amount of accumulated lipid-containing degenerative material, covering a lesion/plaque/area/region, falls above a predetermined threshold value or within a range of values, as measured in terms of a percentage of blockage due to lipid content. If arteries with atheroma lesion(s) having an amount or volume of lipid-containing material above the threshold percentage value or that fall within a range of values are not identified, an alternative treatment process (which may include no treatment or physical intervention) is determined by the physician, in step 110b. If arteries with lipid-containing atheroma lesion/plaque/area/region(s) having an amount or volume of lipid blockage above the predetermined threshold percentage or within a predetermined range of percentages are identified, the patient is then subjected to the delipidation process, in step 110a. The delipidation process of the present specification is described in greater detail below.

At step 108b, a physician determines whether, based on the FFR measurement, blood oxygen delivery is impeded below a threshold value or within a range of values (which is expressed as the maximal flow of blood down a vessel in the presence of stenosis compared to the maximal flow in the hypothetical absence of stenosis). If blood oxygen delivery is impeded below a threshold value or within a predetermined range of values, then in step 112a, a physician treats with physical intervention, such as a stent. In step 112b, if it is determined that blood oxygen delivery is not impeded below a threshold value or does not fall within a predetermined range of values, the physician explores an alternate treatment option (which may include no treatment or the delipidation process of the present specification). In an embodiment, the threshold value is 80%. In an embodiment, the range of values is 1%-79%.

At step 108c, a physician determines whether both the accumulated lipid-containing degenerative material, covering a lesion/plaque/area/region(s) is of an amount or volume falling within a predetermined range of percentages and blood oxygen delivery is impeded as determined by a predetermine range of percentages. If both conditions are met, in step 114a, the physician treats those areas identified as ischemic areas (FFR measurement in a range of 1% to 79%, and preferably below 80%) with a stent implant procedure and subsequently, the remaining areas with the delipidation process of the present specification. In step 114b, if both threshold conditions are not met, then the physician determines if either one of the conditions or neither condition is met and determines an appropriate course of treatment as outlined above.

In an example case, where the analysis from the imaging determines a FFR in the range of 1%-79%, and blockage due to lipids anywhere from 1 to 100%, a physician may decide to physically intervene to improve the blood flow as measured by FFR, and thus, blood oxygen delivery. In an embodiment, the physical intervention is performed by surgically embedding a stent in order to increase the rate of blood flow in the identified atheroma area.

In another example, where the analysis from the imaging determines a FFR in the range of 80%-100%, and blockage due to lipids to be in the range of 20%-70%, the physician may opt for treatment methods that remove or reduce the lipids. In this example, embodiments of the present specification that enable selective modification of HDL particles are utilized.

In yet another example, where the FFR is determined to be in a range of 1% to 79%, and preferably less than 80%, and blockage due to lipids is in the range of 20%-70%, the physician may opt to proceed with the surgical process of embedding a stent. It should be appreciated that when a percentage blockage is stated, such as 20%-70%, it means that a cross-sectional area of a vessel is blocked with lipid containing material and that such blockage occupies a range of 20% to 70% of the cross-sectional area of the vessel.

If arteries with lipid-containing atheroma lesion/plaque/area/region(s) having an amount or volume of lipid blockage within a predetermined range of percentages are identified in step 110a, the patient is then subjected to the delipidation process. In this case, at step 120, a blood fraction of the patient is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

Blood fractionation is known to persons of ordinary skill in the art, and is performed remotely from the method described in context of FIG. 1A. During the fractionation, the blood can optionally be combined with an anticoagulant, such as sodium citrate, and centrifuged at forces approximately equal to 2,000 times gravity. The red blood cells are then aspirated from the plasma. Subsequent to fractionation, the cells are returned to the patient. In some alternate embodiments, Low Density Lipoprotein (LDL) is also separated from the plasma. Separated LDL is usually discarded. In alternative embodiments, LDL is retained in the plasma. In accordance with embodiments of the present specification, blood fraction obtained at 120 includes plasma with High Density Lipoprotein (HDL), and may or may not include other protein particles. In embodiments, autologous plasma collected from the patient is subsequently treated via an approved plasmapheresis device. The plasma may be transported using a continuous or batch process.

At step 122, the blood fraction obtained at 120 is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. Acceptable ratios of solvent to plasma include any combination of solvent and plasma. In some embodiments, ratios used are 2 parts plasma to 1 part solvent, 1 part plasma to 1 part solvent, or 1 part plasma to 2 parts solvent. In an embodiment, when using a solvent comprising 95 parts sevoflurane to 5 parts n-butanol, a ratio of two parts solvent per one part plasma is used. Additionally, in an embodiment employing a solvent containing n-butanol, the present specification uses a ratio of solvent to plasma that yields at least 3% n-butanol in the final solvent/plasma mixture. In an embodiment, a final concentration of n-butanol in the final solvent/plasma mixture is 3.33%. The plasma and solvent are introduced into at least one apparatus for mixing, agitating, or otherwise contacting the plasma with the solvent. The plasma may be transported using a continuous or batch process. Further, various sensing means may be included to monitor pressures, temperatures, flow rates, solvent levels, and the like. The solvents dissolve lipids from the plasma. In embodiments of the present specification, the solvents dissolve lipids to yield treated plasma that contains modified HDL particles with reduced lipid content. The process is designed such that HDL particles are treated to reduce their lipid levels and yield modified HDL particles without destruction of plasma proteins or substantially affecting LDL particles.

Energy is introduced into the system in the form of varied mixing methods, time, and speed. At 124, bulk solvents are removed from the modified HDL particles via centrifugation. In embodiments, any remaining soluble solvent is removed via charcoal adsorption, evaporation, or Hollow Fiber Contractors (HFC) pervaporation. The mixture is optionally tested for residual solvent via use of chromatography (GC), or similar means. The test for residual solvent may optionally be eliminated based on statistical validation.

At 126, the treated plasma containing modified HDL particles with reduced lipid content, which was separated from the solvents at 124, is treated appropriately and subsequently returned to the patient. The modified HDL particles are HDL particles with an increased concentration of pre-beta HDL. Concentration of pre-beta HDL is greater in the modified HDL, relative to the original HDL that was present in the plasma before treating it with the solvent. The resulting treated plasma containing the HDL particles with reduced lipid and increased pre-beta concentration is optionally combined with the patient's red blood cells, if the red cells were not already returned during plasmapheresis, and administered to the patient. One route of administration is through the vascular system, preferably intravenously.

In embodiments, the patient is monitored again for changes in the previously monitored atheroma areas and volumes, specifically for lipid-containing degenerative material. Therefore the process is repeated from step 102, as described above. In embodiments, the patient is monitored repeatedly within a period of three to six months. The treatment cycle is also repeated at this frequency until the monitoring suggests substantially or completely enhanced cholesterol efflux. In an embodiment, when the atheroma area and volume are monitored to be below threshold, the patient may be considered to have been treated and may not require further repetition of the treatment cycle. In some embodiments, frequency of treatment may vary depending on the volume to be treated and the severity of the condition of the patient.

### Atheroembolic Renal Disease

Renal Arterial Stenosis (RAS) is a systemic disease and patients may have multiple lesions throughout their vasculature. Sometimes, the plaque within the arteries may break away and damage kidneys, resulting in Atheroembolic Renal Disease (AERD). Therefore, it should be noted herein that the treatment methods of the present specification are not implemented based on an overall patient health-based treatment strategy, but rather a “lesion/plaque/area/region”-based treatment strategy.

FIG. 1B is a flow chart illustrating another exemplary process of treating cholesterol-related diseases, such as, but not limited to Atheroembolic Renal Disease (AERD), in accordance with some embodiments of the present specification. In all cases, a patient first presents with renal arterial stenosis—a blockage in an artery that supplies blood to the kidney. At step 132, it is determined whether a patient has elevated Blood Pressure (BP). Recent onset of hypertension may be a clinical manifestation of the presence of plaque. If it is determined that the patient has High BP (HBP), the physician, may look for atheroembolic renal disease (AERD) at step 134. While AERD may not cause any symptoms, some of the following symptoms may appear slowly and worsen over time: blood in the urine, fever, muscle aches, headache, weight loss, foot pain or blue toes, nausea, among other symptoms. If AERD is not identified, then at 136, a stent is placed in the patient to reverse any blockage that may be resulting in HBP.

If, at 134, AERD is identified in addition to elevated BP, then the physician may place a stent at step 138 in order to reverse blockage and elevations in BP. Additionally, at step 140, the physician may determine whether the procedure of placing a stent has worked to address both elevated BP levels and AERD. If not, an additional stent may be placed, or the delipidation process, in accordance with embodiments of the present specification and described with respect to FIG. 1A, may be used. The treatment decision may be based on “lesion/plaque/area/region” determination.

At step 132, if it is determined that the patient has normal levels of BP, the physician may still check for symptoms or signs of AERD at step 142. The check may be conducted on the basis of symptoms such as, but not limited to, blindness, blood in the urine, fever, muscle aches, headache, weight loss, foot pain or blue toes, nausea, among other symptoms. If, at 142, AERD is not detected, then, at step 144, the physician may determine an appropriate course of treatment, based on the symptoms and any other diagnosis. If there is renal stenosis (the presence of cholesterol-containing plaque) absent both elevated HBP and AERD, then the physician may opt to follow the procedure outlined above in context of FIG. 1A for cardiovascular diseases, which can result in either one or both of a stent and/or the delipidation process of the present specification.

If the patient is diagnosed with AERD but has normal BP levels, then the physician may proceed to step 146, and the subject or the patient is monitored for one or more atheroma areas and/or volumes via a diagnostic procedure to determine the cause of renal dysfunction, and the extent of renal arterial stenosis. In an embodiment, advanced medical imaging techniques, such as, but not limited to Computer Tomography (CT) angiogram and/or Intravascular Ultrasound (IVUS) and/or Near IR spectroscopy, may be used to detect areas within the inner layer of artery walls where lipid-containing degenerative material may have accumulated. Accumulated degenerative material may include fatty deposits which may include mostly macrophage cells, or debris, containing lipids, calcium and a variable amount of fibrous connective tissue. Analysis from the imaging techniques may also be used to identify and therefore monitor volumes of lipid-containing degenerative material accumulated within the inner layer of artery walls. Lipid-containing degenerative material and non-lipid-containing degenerative material may swell in the artery wall, thereby intruding into the channel of the artery and narrowing it, resulting in restricting of blood flow and causing renal abnormalities.

Based on analysis from the diagnostic technique, the presence and type of degenerative material is confirmed, the extent or percentage of degenerative material (lipid-containing or non-lipid-containing) is determined, and the extent of blood oxygen delivery based on Fractional Flow Reserve (FFR) is identified. The process is stopped if no degenerative material is detected, or if the level of degenerative material is below a predetermined threshold or falls outside of a predetermined range of values. In an embodiment, the physician identifies one or more renal arteries with stenosis that have a blockage of 20%-70% due to accumulated lipids, in order to implement treatment methods in accordance with the present specification. In an embodiment, FFR is used to measure pressure differences across arterial stenosis to determine the likelihood that the stenosis impedes blood flow, and thus, oxygen delivery to the kidney (ischemia).

Different types of treatments may be provided depending on the diagnostic results and threshold values. At this stage, the physician may determine that either the treatment in accordance with embodiments of the present specification is not required as the disease has subsided, is not present, is not sufficient, or has been treated; or an alternative form of treatment is required.

Referring back to FIG. 1C, the table compares different types of treatments that may be administered for combinations of various ranges of a Fractional Flow Reserve (FFR) 402, which is indicative of a change in rate of flow of blood associated with a blockage (and thus blood oxygen delivery), provided in terms of percentage of FFR, and various ranges of blockage due to lipid content 404, provided in terms of percentage of blockage due to lipid content. Referring to the table, each cell, such as cells 406, correspond to a combination of a range 402 (indicative of FFR) and a range 404 (indicative of the percentage or extent of blockage due to lipid content), which further indicates at least one method of treatment that may be suitable for that combination.

In embodiments, the different types of treatments are coded as A, B, C, and D. Treatment type ‘A’ corresponds to an invasive treatment process where a stent is embedded through physical intervention. Treatment type ‘B’ corresponds to implementing the treatment methods of selectively modifying HDL particles, in accordance with the embodiments of the present specification. In an embodiment, it is preferable to selectively modify HDL particles (and perform the HDL infusions) where the Fractional Flow Reserve (FFR) ranges from 80-100% and the accumulated lipid obstruction ranges from 20-70%, as noted by sections 404. It should be noted herein that in embodiments, a FFR of 1-79% represents an ischemic condition, wherein 80-100% FFR represents a non-ischemic condition. In most cases, treatment types ‘A’ and/or ‘B’ may be able to address the condition. Treatment type ‘D’ corresponds to cases where neither of the stated treatment types (A, B, or C) is required. In some cells, such as cells 408, two treatment options may be indicated and the physician would decide upon the appropriate course of treatment.

Treatment type ‘C’ corresponds to cases where a combination of both a stent as well as selective modification of HDL particles is administered. Renal Arterial Stenosis (RAS) is a systemic disease and patients may have multiple lesions throughout their vasculature. It should be noted herein that the treatment methods of the present specification are not implemented based on an overall patient health-based treatment strategy, but rather a “lesion/plaque/area/region”-based treatment strategy. Thus, in a few cases, a physician may decide to combine the treatments and administer treatment type ‘C’. If, in a particular patient, one or more areas or lesions have a FFR percentage measured at 79% or less, then those areas would have a stent implanted. If the same patient presents with additional, remaining lesions that exhibit lipid-based blockage in the range of 20-70% and also an FFR of 80-100%, then the patient would undergo a subsequent delipidation. Therefore, both interventions may be used for patients having multiple lesions with different levels of disease at each lesion.

The physician determines whether the amount of accumulated lipid-containing degenerative material, covering a lesion/plaque/area/region, falls above or below a predetermined threshold percentage or within a predetermined range of percentages, as measured in terms of a percentage of blockage due to lipid content. If arteries with lipid-containing atheroma lesion(s) having an amount or volume above or below a threshold percentage or falling within a predetermined range of percentages are not identified, an alternative treatment process (which may include no treatment or physical intervention) is determined by the physician. If arteries with lipid-containing atheroma lesion/plaque/area/region(s) having an amount or volume of lipid blockage falling within a predetermined range of percentages are identified, the patient is then subjected to the delipidation process. The delipidation process of the present specification is described in greater detail with respect to FIG. 1A.

The physician also determines whether, based on the FFR measurement, blood oxygen delivery is impeded below a threshold value or falls within a range of values (which is expressed as the maximal flow of blood down a vessel in the presence of stenosis compared to the maximal flow in the hypothetical absence of stenosis). If blood oxygen delivery is impeded below a threshold value or falls within a range of values, a physician treats with physical intervention, such as a stent. If it is determined that blood oxygen delivery is not impeded above a threshold value, the physician explores an alternate treatment option (which may include no treatment or the delipidation process of the present specification). In an embodiment, the threshold value is 80%. In an embodiment, the range of values is 1%-79%.

Subsequently, a physician determines whether both the accumulated lipid-containing degenerative material, covering a lesion/plaque/area/region(s) is in an amount or volume within a predetermined range of percentages and blood oxygen delivery is impeded above a threshold value or within a predetermined range of values. If both threshold conditions are met, the physician treats those areas identified as ischemic areas (FFR below 80%, or within a range of 1% to 79%) with a stent implant procedure and subsequently, the remaining areas with the delipidation process of the present specification. If both threshold conditions are not met, then the physician determines if either one of the conditions or neither condition is met and determines an appropriate course of treatment as outlined above.

In an example case, where the analysis from the imaging determines a FFR in the range of 1%-79%, and blockage due to lipids anywhere from 1 to 100%, a physician may decide to physically intervene to improve blood oxygen delivery, as measured by FFR. In an embodiment, the physical intervention is performed by surgically embedding a stent in order to increase the rate of blood flow in the identified atheroma area.

In another example, where the analysis from the imaging determines a FFR in the range of 80%-100%, and blockage due to lipids to be in the range of 20%-70%, the physician may opt for treatment methods that remove or reduce the lipids. In this example, embodiments of the present specification that enable selective modification of HDL particles are utilized. In yet another example, where the FFR is determined to be less than 80% (in a range of 1% to 79%), and blockage due to lipids is in the range of 20%-70%, the physician may opt to proceed with the surgical process of embedding a stent. It should be appreciated that when a percentage blockage is stated, such as 20%-70%, it means that a cross-sectional area of a vessel is blocked with lipid containing material and that such blockage occupies a range of 20% to 70% of the cross-sectional area of the vessel.

If arteries with lipid-containing atheroma area/volume within a predetermined range of percentages are identified, the patient is then subjected to the delipidation process. In this case, a blood fraction of the patient is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

Blood fractionation is known to persons of ordinary skill in the art, and is performed remotely from the method described in context of FIG. 1A. During the fractionation, the blood can optionally be combined with an anticoagulant, such as sodium citrate, and centrifuged at forces approximately equal to 2,000 times gravity. The red blood cells are then aspirated from the plasma. Subsequent to fractionation, the cells are returned to the patient. In some alternate embodiments, Low Density Lipoprotein (LDL) is also separated from the plasma. Separated LDL is usually discarded. In alternative embodiments, LDL is retained in the plasma. In accordance with embodiments of the present specification, obtained blood fraction includes plasma with High Density Lipoprotein (HDL), and may or may not include other protein particles. In embodiments, autologous plasma collected from the patient is subsequently treated via an approved plasmapheresis device. The plasma may be transported using a continuous or batch process.

The blood fraction obtained is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. Acceptable ratios of solvent to plasma include any combination of solvent and plasma. In some embodiments, ratios used are 2 parts plasma to 1 part solvent, 1 part plasma to 1 part solvent, or 1 part plasma to 2 parts solvent. In an embodiment, when using a solvent comprising 95 parts sevoflurane to 5 parts n-butanol, a ratio of two parts solvent per one part plasma is used. Additionally, in an embodiment employing a solvent containing n-butanol, the present specification uses a ratio of solvent to plasma that yields at least 3% n-butanol in the final solvent/plasma mixture. In an embodiment, a final concentration of n-butanol in the final solvent/plasma mixture is 3.33%. The plasma and solvent are introduced into at least one apparatus for mixing, agitating, or otherwise contacting the plasma with the solvent. The plasma may be transported using a continuous or batch process. Further, various sensing means may be included to monitor pressures, temperatures, flow rates, solvent levels, and the like. The solvents dissolve lipids from the plasma. In embodiments of the present specification, the solvents dissolve lipids to yield treated plasma that contains modified HDL particles with reduced lipid content. The process is designed such that HDL particles are treated to reduce their lipid levels and yield modified HDL particles without destruction of plasma proteins or substantially affecting LDL particles.

Energy is introduced into the system in the form of varied mixing methods, time, and speed. Bulk solvents are removed from the modified HDL particles via centrifugation. In embodiments, any remaining soluble solvent is removed via charcoal adsorption, evaporation, or Hollow Fiber Contractors (HFC) pervaporation. The mixture is optionally tested for residual solvent via use of chromatography (GC), or similar means. The test for residual solvent may optionally be eliminated based on statistical validation.

The treated plasma containing modified HDL particles with reduced lipid content, which was separated from the solvents, is treated appropriately and subsequently returned to the patient. The modified HDL particles are HDL particles with an increased concentration of pre-beta HDL. Concentration of pre-beta HDL is greater in the modified HDL, relative to the original HDL that was present in the plasma before treating it with the solvent. The resulting treated plasma containing the HDL particles with reduced lipid and increased pre-beta concentration is optionally combined with the patient's red blood cells, if the red cells were not already returned during plasmapheresis, and administered to the patient. One route of administration is through the vascular system, preferably intravenously.

In embodiments, the patient is monitored again for changes in the previously monitored atheroma areas and volumes, specifically for lipid-containing degenerative material. Therefore the process is repeated, as described above. In embodiments, the patient is monitored repeatedly within a period of three to six months. The treatment cycle is also repeated at this frequency until the monitoring suggests substantially or completely enhanced cholesterol efflux. In an embodiment, when the atheroma area and volume are monitored to be below threshold, the patient may be considered to have been treated and may not require further repetition of the treatment cycle. In some embodiments, frequency of treatment may vary depending on the volume to be treated and the severity of the condition of the patient.

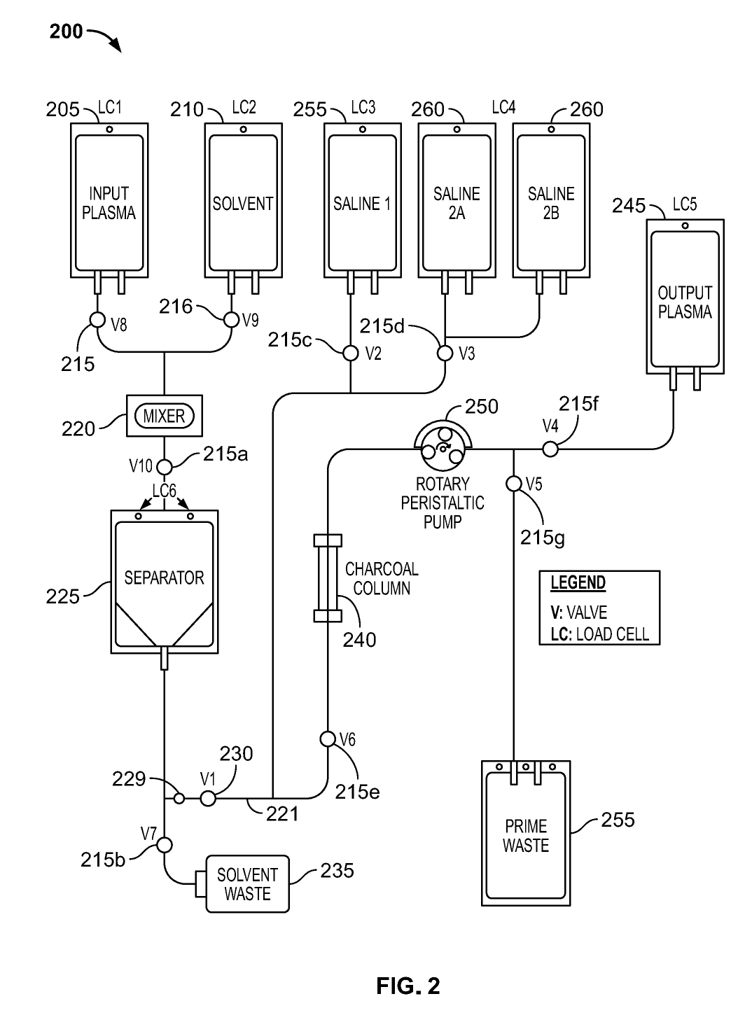

FIG. 2 illustrates an exemplary embodiment of a system and its components used to achieve the methods of the present specification. The figure depicts an exemplary basic component flow diagram defining elements of the HDL modification system 200. Embodiments of the components of system 200 are utilized after obtaining a blood fraction from a patient or another individual (donor). The plasma, separated from the blood is brought in a sterile bag to system 200 for further processing. The plasma may be separated from blood using a known plasmapheresis device. The plasma may be collected from the patient into a sterile bag using standard apheresis techniques. The plasma is then brought in the form of a fluid input to system 200 for further processing. In embodiments, system 200 is not connected to the patient at any time and is a discrete, stand-along system for delipidating plasma. The patient's plasma is processed by system 200 and brought back to the patient's location to be reinfused back into the patient. In alternate embodiments, the system may be a continuous flow system that is connected to the patient in which both plasmapheresis and delipidation are performed in an excorporeal, parallel system and the delipidated plasma product is returned to the patient.

A fluid input 205 (containing blood plasma) is provided and connected via tubing to a mixing device 220. A solvent input 210 is provided and also connected via tubing to mixing device 220. In embodiments, valves 215, 216 are used to control the flow of fluid from fluid input 205 and solvent from solvent input 210 respectively. It should be appreciated that the fluid input 205 contains any fluid that includes HDL particles, including plasma having LDL particles or devoid of LDL particles, as discussed above. It should further be appreciated that solvent input 210 can include a single solvent, a mixture of solvents, or a plurality of different solvents that are mixed at the point of solvent input 210. While depicted as a single solvent container, solvent input 210 can comprise a plurality of separate solvent containers. Embodiments of types of solvents that may be used are discussed above.

Mixer 220 mixes fluid from fluid input 205 and solvent from solvent input 210 to yield a fluid-solvent mixture. In embodiments, mixer 220 is capable of using a shaker bag mixing method with the input fluid and input solvent in a plurality of batches, such as 1, 2, 3 or more batches. An exemplary mixer is a Barnstead Labline orbital shaker table. In alternative embodiments, other known methods of mixing are utilized. Once formed, the fluid-solvent mixture is directed, through tubing and controlled by at least one valve 215a, to a separator 225. In an embodiment, separator 225 is capable of performing bulk solvent separation through gravity separation in a funnel-shaped bag.

In separator 225, the fluid-solvent mixture separates into a first layer and second layer. The first layer comprises a mixture of solvent and lipid that has been removed from the HDL particles. The first layer is transported through a valve 215b to a first waste container 235. The second layer comprises a mixture of residual solvent, modified HDL particles, and other elements of the input fluid. One of ordinary skill in the art would appreciate that the composition of the first layer and the second layer would differ based upon the nature of the input fluid. Once the first and second layers separate in separator 225, the second layer is transported through tubing to a solvent extraction device 240. In an embodiment, a pressure sensor 229 and valve 230 is positioned in the flow stream to control the flow of the second layer to solvent extraction device 240.

The opening and closing of valves 215, 216 to enable the flow of fluid from input containers 205, 210 may be timed using mass balance calculations derived from weight determinations of the fluid inputs 205, 210 and separator 225. For example, the valve 215b between separator 225 and first waste container 235 and valve 230 between separator 225 and solvent extraction device 240 open after the input masses (fluid and solvent) substantially balances with the mass in separator 225 and a sufficient period of time has elapsed to permit separation between the first and second layers. Depending on what solvent is used, and therefore which layer settles to the bottom of separator 225, either valve 215b between separator 225 and first waste container 235 is opened or valve 230 between separator 225 and solvent extraction device 240 is opened. One of ordinary skill in the art would appreciate that the timing of the opening is dependent upon how much fluid is in the first and second layers and would further appreciate that it is preferred to keep valve 215b between separator 225 and first waste container 235 open just long enough to remove all of the first layer and some of the second layer, thereby ensuring that as much solvent as possible has been removed from the fluid being sent to solvent extraction device 240.

In embodiments, an infusion grade fluid (“IGF”) may be employed via one or more inputs 260 which are in fluid communication with the fluid path 221 leading from separator 225 to solvent extraction device 240 for priming. In an embodiment, saline is employed as the infusion grade priming fluid in at least one of inputs 260. In an embodiment, 0.9% sodium chloride (saline) is employed. In other embodiments, glucose may be employed as the infusion grade priming fluid in any one of inputs 260.

A plurality of valves 215c and 215d are also be incorporated in the flow stream from glucose input 255 and saline input 260 respectively, to the tubing providing the flow path 221 from separator 225 to solvent extraction device 240. IGF such as saline and/or glucose are incorporated into embodiments of the present specification in order to prime solvent extraction device 240 prior to operation of the system. In embodiments, saline is used to prime most of the fluid communication lines and solvent extraction device 240. If priming is not required, the IGF inputs are not employed. Where such priming is not required, the glucose and saline inputs are not required. Also, one of ordinary skill in the art would appreciate that the glucose and saline inputs can be replaced with other primers if required by the solvent extraction device 240 requires it.

In some embodiments, solvent extraction device 240 is a charcoal column designed to remove the specific solvent used in solvent input 210. An exemplary solvent extraction device 240 is an Asahi Hemosorber charcoal column, or the Bazter/Gambro Adsorba 300C charcoal column or any other charcoal column that is employed in blood hemoglobin perfusion procedures. A pump 250 is used to move the second layer from separator 225, through solvent extraction device 240, and to an output container 245. In embodiments, pump 250 is a rotary peristaltic pump, such as a Masterflex Model 77201-62.

The first layer is directed to waste container 235 that is in fluid communication with separator 225 through tubing and at least one valve 215b. Additionally, other waste, if generated, can be directed from the fluid path connecting solvent extraction device 240 and output container 245 to a second waste container 255. Optionally, in an embodiment, a valve 215f is included in the path from the solvent extraction device 240 to the output container 245. Optionally, in an embodiment, a valve 215g is included in the path from the solvent extraction device 240 to the second waste container 255.

In an embodiment of the present specification, gravity is used, wherever practical, to move fluid through each of the plurality of components. For example, gravity is used to drain input plasma 205 and input solvent 210 into mixer 220. Where mixer 220 comprises a shaker bag and separator 225 comprises a funnel bag, fluid is moved from the shaker bag to the funnel bag and, subsequently, to first waste container 235, if appropriate, using gravity.

In an additional embodiment, not shown in FIG. 2, the output fluid in output container 245 is subjected to a solvent detection system, or lipid removing agent detection system, to determine if any solvent, or other undesirable component, is in the output fluid. In embodiments, a solvent sensor is only employed in a continuous flow system. In one embodiment, the output fluid is subjected to sensors that are capable of determining the concentrations of solvents introduced in the solvent input, such as n-butanol or di-isopropyl ether. The output fluid is returned to the bloodstream of the patient and the solvent concentrations must be below a predetermined level to carry out this operation safely. In embodiments, the sensors are capable of providing such concentration information on a real-time basis and without having to physically transport a sample of the output fluid, or air in the headspace, to a remote device. The resultant separated modified HDL particles are then introduced to the bloodstream of the patient.

In one embodiment, molecularly imprinted polymer technology is used to enable surface acoustic wave sensors. A surface acoustic wave sensor receives an input, through some interaction of its surface with the surrounding environment, and yields an electrical response, generated by the piezoelectric properties of the sensor substrate. To enable the interaction, molecularly imprinted polymer technology is used. Molecularly imprinted polymers are plastics programmed to recognize target molecules, like pharmaceuticals, toxins or environmental pollutants, in complex biological samples. The molecular imprinting technology is enabled by the polymerization of one or more functional monomers with an excess of a crosslinking monomer in presence of a target template molecule exhibiting a structure similar to the target molecule that is to be recognized, i.e. the target solvent.

The use of molecularly imprinted polymer technology to enable surface acoustic wave sensors can be made more specific to the concentrations of targeted solvents and are capable of differentiating such targeted solvents from other possible interferents. As a result, the presence of acceptable interferents that may have similar structures and/or properties to the targeted solvents would not prevent the sensor from accurately reporting existing respective solvent concentrations.

Alternatively, if the input solvent comprises certain solvents, such as n-butanol, electrochemical oxidation could be used to measure the solvent concentration. Electrochemical measurements have several advantages. They are simple, sensitive, fast, and have a wide dynamic range. The instrumentation is simple and not affected by humidity. In one embodiment, the target solvent, such as n-butanol, is oxidized on a platinum electrode using cyclic voltammetry. This technique is based on varying the applied potential at a working electrode in both the forward and reverse directions, at a predefined scan rate, while monitoring the current. One full cycle, a partial cycle, or a series of cycles can be performed. While platinum is the preferred electrode material, other electrodes, such as gold, silver, iridium, or graphite, could be used. Although, cyclic voltammetric techniques are used, other pulse techniques such as differential pulse voltammetry or square wave voltammetry may increase the speed and sensitivity of measurements.

Embodiments of the present specification expressly cover any and all forms of automatically sampling and measuring, detecting, and analyzing an output fluid, or the headspace above the output fluid. For example, such automated detection can be achieved by integrating a mini-gas chromatography (GC) measuring device that automatically samples air in the output container, transmits it to a GC device optimized for the specific solvents used in the delipidation process, and, using known GC techniques, analyzes the sample for the presence of the solvents.

Referring back to FIG. 2, suitable materials for use in any of the apparatus components as described herein include materials that are biocompatible, approved for medical applications that involve contact with internal body fluids, and in compliance with U.S. PVI or ISO 10993 standards. Further, the materials do not substantially degrade from, for instance, exposure to the solvents used in the present specification, during at least a single use. The materials are sterilizable either by radiation or ethylene oxide (EtO) sterilization. Such suitable materials are capable of being formed into objects using conventional processes, such as, but not limited to, extrusion, injection molding and others. Materials meeting these requirements include, but are not limited to, nylon, polypropylene, polycarbonate, acrylic, polysulfone, polyvinylidene fluoride (PVDF), fluoroelastomers such as VITON, available from DuPont Dow Elastomers L.L.C., thermoplastic elastomers such as SANTOPRENE, available from Monsanto, polyurethane, polyvinyl chloride (PVC), polytetrafluoroethylene (PTFE), polyphenylene ether (PFE), perfluoroalkoxy copolymer (PFA), which is available as TEFLON PFA from E.I. du Pont de Nemours and Company, and combinations thereof.

Valves 215, 215a, 215b, 215c, 215d, 215e, 215f, 215g, 216 and any other valve used in each embodiment may be composed of, but are not limited to, pinch, globe, ball, gate or other conventional valves. In some embodiments, the valves are occlusion valves such as Acro Associates' Model 955 valve. However, the present specification is not limited to a valve having a particular style. Further, the components of each system described in accordance with embodiments of the present specification may be physically coupled together or coupled together using conduits that may be composed of flexible or rigid pipe, tubing or other such devices known to those of ordinary skill in the art.

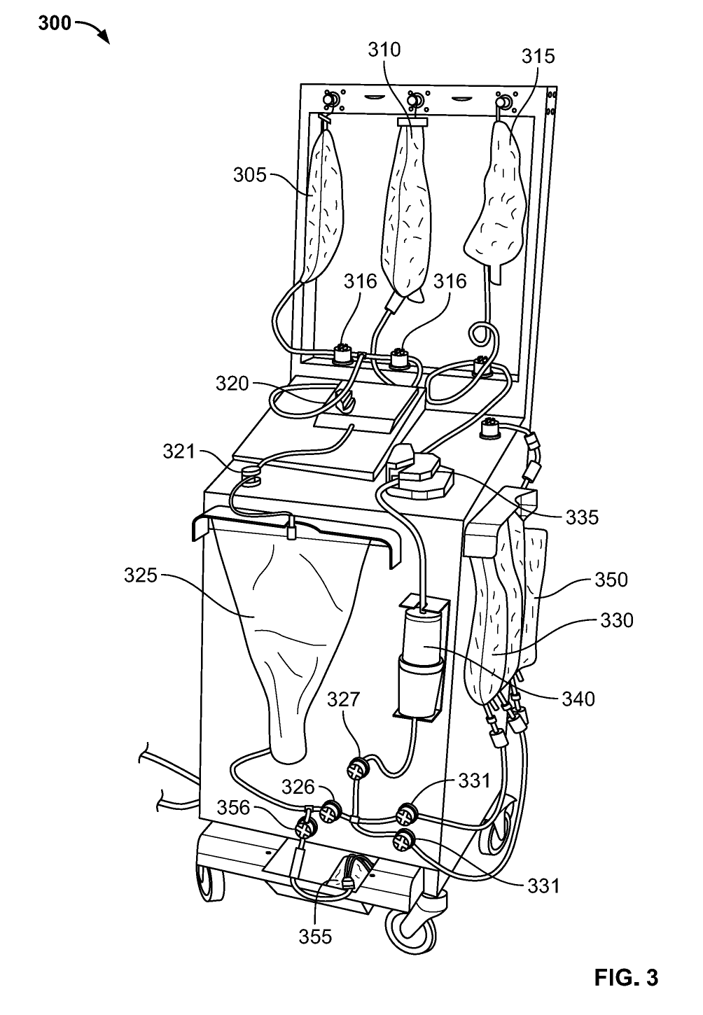

FIG. 3 illustrates an exemplary configuration of a system used in accordance with some embodiments of the present specification to achieve the processes disclosed herein. Referring to FIG. 3, a configuration of basic components of the HDL modification system 300 is shown. A fluid input 305 is provided and connected via tubing to a mixing device 320. A solvent input 310 is provided and also connected via tubing to a mixing device 320. Preferably valves 316 are used to control the flow of fluid from fluid input 305 and solvent from solvent input 310. It should be appreciated that the fluid input 305 preferably contains any fluid that includes HDL particles, including plasma having LDL particles or devoid of LDL particles, as discussed above. It should further be appreciated that solvent input 310 can include a single solvent, a mixture of solvents, or a plurality of different solvents that are mixed at the point of solvent input 310. While depicted as a single solvent container, solvent input 310 can comprise a plurality of separate solvent containers. The types of solvents that are used and preferred are discussed above.

The mixer 320 mixes fluid from fluid input 305 and solvent from solvent input 310 to yield a fluid-solvent mixture. Preferably, mixer 320 is capable of using a shaker bag mixing method with the input fluid and input solvent in a plurality of batches, such as 1, 2, 3 or more batches. Once formed, the fluid-solvent mixture is directed, through tubing and controlled by at least one valve 321, to a separator 325. In a preferred embodiment, separator 325 is capable of performing bulk solvent separation through gravity separation in a funnel-shaped bag.

In the separator 325, the fluid-solvent mixture separates into a first layer and second layer. The first layer comprises a mixture of solvent and lipid that has been removed from the HDL particles. The second layer comprises a mixture of residual solvent, modified HDL particles, and other elements of the input fluid. One of ordinary skill in the art would appreciate that the composition of the first layer and the second layer would differ based upon the nature of the input fluid. Once the first and second layers separate in separator 325, the second layer is transported through tubing to a solvent extraction device 340. Preferably, a pressure sensor 326 and valve 327 is positioned in the flow stream to control the flow of the second layer to the solvent extraction device 340.

Preferably, a glucose input 330 and saline input 350 is in fluid communication with the fluid path leading from the separator 325 to the solvent extraction device 340. A plurality of valves 331 is also preferably incorporated in the flow stream from the glucose input 330 and saline input 350 to the tubing providing the flow path from the separator 325 to the solvent extraction device 340. Glucose and saline are incorporated into the present specification in order to prime the solvent extraction device 340 prior to operation of the system. Where such priming is not required, the glucose and saline inputs are not required. Also, one of ordinary skill in the art would appreciate that the glucose and saline inputs can be replaced with other primers if the solvent extraction device 340 requires it.

The solvent extraction device 340 is preferably a charcoal column designed to remove the specific solvent used in the solvent input 310. An exemplary solvent extraction device 340 is an

Asahi Hemosorber charcoal column. A pump 335 is used to move the second layer from the separator 325, through the solvent extraction device 340, and to an output container 315. The pump is preferably a peristaltic pump, such as a Masterflex Model 77201-62.

The first layer is directed to a waste container 355 that is in fluid communication with separator 325 through tubing and at least one valve 356. Additionally, other waste, if generated, can be directed from the fluid path connecting solvent extraction device 340 and output container 315 to waste container 355.

Preferably, an embodiment of the present specification uses gravity, wherever practical, to move fluid through each of the plurality of components. For example, preferably gravity is used to drain the input plasma 305 and input solvent 310 into the mixer 320. Where the mixer 320 comprises a shaker bag and separator 325 comprises a funnel bag, fluid is moved from the shaker bag to the funnel bag and, subsequently, to the waste container 355, if appropriate, using gravity.

In general, the present specification preferably comprises configurations wherein all inputs, such as input plasma and input solvents, disposable elements, such as mixing bags, separator bags, waste bags, solvent extraction devices, and solvent detection devices, and output containers are in easily accessible positions and can be readily removed and replaced by a technician.

To enable the operation of the above described embodiments of the present specification, it is preferable to supply a user of such embodiments with a packaged set of components, in kit form, comprising each component required to practice embodiments of the present specification. The kit may include an input fluid container (i.e. a high density lipoprotein source container), a lipid removing agent source container (i.e. a solvent container), disposable components of a mixer, such as a bag or other container, disposable components of a separator, such as a bag or other container, disposable components of a solvent extraction device (i.e. a charcoal column), an output container, disposable components of a waste container, such as a bag or other container, solvent detection devices, and, a plurality of tubing and a plurality of valves for controlling the flow of input fluid (high density lipoprotein) from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container.

In one embodiment, a kit comprises a plastic container having disposable components of a mixer, such as a bag or other container, disposable components of a separator, such as a bag or other container, disposable components of a waste container, such as a bag or other container, and, a plurality of tubing and a plurality of valves for controlling the flow of input fluid (high density lipoprotein) from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container. Disposable components of a solvent extraction device (i.e. a charcoal column), the input fluid, the input solvent, and solvent extraction devices may be provided separately.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method for treatment of cardiovascular disease in a patient, comprising:
monitoring changes in one or more blood vessels in the patient;
based on said monitoring, determining if lipid-containing degenerative material is present in said one or more blood vessels;
monitoring a degree of blood oxygen delivery;
based on said determination of lipid-containing degenerative material and degree of blood oxygen delivery, determining a treatment protocol for said cardiovascular disease, wherein the treatment protocol comprises at least one of a placement of a stent in the patient, an administration to the patient of a composition derived from mixing a blood fraction of the patient with a lipid removing agent, or a placement of a stent in the patient together with an administration to the patient of a composition derived from mixing a blood fraction of the patient with a lipid removing agent.

2. The method of claim 1, wherein the composition is derived by
obtaining the blood fraction from the patient;
mixing said blood fraction with the lipid removing agent to yield modified high-density lipoproteins;
separating said modified high-density lipoproteins; and
delivering said modified high-density lipoproteins to said patient.

3. The method of claim 1, further comprising:
connecting the patient to a device for withdrawing blood;
withdrawing blood from the patient; and
separating blood cells from the blood to yield the blood fraction containing high density lipoproteins and low density lipoproteins.

4. The method of claim 2, wherein the modified high density lipoproteins have an increased concentration of pre-beta high density lipoprotein relative to high density lipoproteins from the blood fraction prior to mixing.

5. The method of claim 1 wherein the degree of blood oxygen delivery is monitored by measuring the patient's fractional flow reserve.

6. The method of claim 5, wherein the treatment protocol is determined to be the placement of the stent in the patient if the patient's fractional flow reserve is within a first range of values.

7. The method of claim 6, wherein the first range of values is 1% to 79%.

8. The method of claim 5, wherein the treatment protocol is determined to be the administration of the composition derived from mixing the blood fraction of the patient with the lipid removing agent if the patient's fractional flow reserve is within a second range of values and if the lipid-containing degenerative material occupies a cross sectional area of the one or more blood vessels that is within a third range of values.

9. The method of claim 8, wherein the second range of values is 80%-100% and wherein the third range of values is 20% to 70%.

10. The method of claim 1 wherein the cardiovascular disease is at least one of homozygous familial hypercholesterolemia, heterozygous familial hypercholesterolemia, ischemic stroke, coronary artery disease, acute coronary syndrome, or peripheral arterial disease.

11. A method for treatment of a lipid-related disease in a patient, comprising:
administering a diagnostic procedure to the patient configured to monitor one or more blood vessels;
determining a presence of lipid-containing degenerative material in the one or more blood vessels;
identifying an extent of the presence of lipid-containing degenerative material and comparing said extent to a predetermined lipid-containing degenerative material range of values;
identifying a level of fractional flow reserve (FFR) and comparing said level to a predetermined threshold FFR range of values;
proceeding with a first treatment protocol if said extent of the presence of lipid-containing degenerative material is within a first range and said FFR level is within a second range;
proceeding with a second treatment protocol if said FFR level is within a third range that is less than the FFR level within a second range;
proceeding with a third treatment protocol if said extent of the presence of lipid-containing degenerative material is within a fourth range that is less than the first range and if said FFR level is within the first range; and
proceeding with a fourth treatment protocol if said extent of the presence of lipid-containing degenerative material is within a fifth range that is greater than the first range and if said FFR level is within the first range, wherein each of the first treatment protocol, second treatment protocol, third treatment protocol, and fourth treatment protocol are different.

12. The method of claim 11, wherein the extent of the presence of lipid-containing degenerative material is within 20% to 70% in a first range.

13. The method of claim 11 wherein said FFR level is within 80% to 100% in a second range.

14. The method of claim 11 wherein said FFR level is within 1% to 79% in a second range.

15. The method of claim 11, wherein the extent of the presence of lipid-containing degenerative material is within 1% to 19% in a fourth range.

16. The method of claim 11, wherein the extent of the presence of lipid-containing degenerative material is within 71% to 100% in a fifth range.

17. The method of claim 11, wherein the first treatment protocol is an administration to the patient a composition derived from mixing a blood fraction of the patient with a lipid removing agent without a placement of a stent in the patient.

18. The method of claim 11, wherein the second treatment protocol is a placement of a stent in the patient without an administration to the patient of a composition derived from mixing a blood fraction of the patient with a lipid removing agent.

19. The method of claim 11, wherein the fourth treatment protocol is selected from either the first treatment protocol or the third protocol, wherein the third protocol is no treatment.

20. The method of claim 17, wherein the composition is derived by:
obtaining the blood fraction from the patient;
mixing said blood fraction with the lipid removing agent to yield modified high-density lipoproteins;
separating said modified high-density lipoproteins; and
delivering said modified high-density lipoproteins to said patient.

21. The method of claim 11, wherein the lipid-related disease is at least one of homozygous familial hypercholesterolemia, heterozygous familial hypercholesterolemia, ischemic stroke, coronary artery disease, acute coronary syndrome, renal arterial stenosis, peripheral arterial disease, or atheroembolic renal disease.
